(19) United States
(12) Patent Application Publication
(10) Pub. No.: US 2007/0167913 A1
(43) Pub. Date: Jul. 19, 2007

(54) VASCULAR SHEATH WITH VARIABLE LUMEN CONSTRUCTION

(75) Inventors: Jeffrey M. Elkins, Novato, CA (US); Harry B. Goodson IV, Fremont, CA (US); Craig A. Ball, San Carlos, CA (US); Aurelio Valencia, East Palo Alto, CA (US); Samir R. Patel, Mountain View, CA (US); Neema Hekmat, Mountain View, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FlowMedica, Inc., Fremont, CA (US)

(21) Appl. No.: 11/548,565

(22) Filed: Oct. 11, 2006

Related U.S. Application Data
(60) Provisional application No. 60/742,579, filed on Dec. 5, 2005.
(60) Provisional application No. 60/725,756, filed on Oct. 11, 2005.

(51) Int. Cl. A61M 5/178; A61F 2/06; A61M 31/00

(52) U.S. Cl. 604/158; 604/164.01; 604/508; 623/1.16

(57) ABSTRACT

A system for delivering a renal treatment and a peripheral treatment includes a renal catheter, a peripheral catheter, and an introducer sheath having first and second lumens. The first lumen is configured to receive the renal catheter and is sized to extend from a patient insertion site to a femoral or iliac artery location near or distal to a patient aortic branch. The second lumen is configured to receive the peripheral catheter and is sized to extend from the patient insertion site to an opposite femoral or iliac artery location near or distal to the patient aortic branch. A method of delivering a renal treatment and a peripheral treatment includes positioning an introducer sheath in an iliac artery, advancing a renal catheter and a peripheral catheter through the introducer sheath, where the renal catheter is separated from the peripheral catheter within the introducer sheath by a flap.

### CROSS-REFERENCES TO RELATED APPLICATIONS

This application claims the benefit of U.S. Provisional Patent Application Nos. 60/725,756 filed Oct. 11, 2005 and 60/742,579 filed Dec. 5, 2005, the entire contents of which are incorporated herein by reference for all purposes. This application is also related to U.S. patent application Ser. Nos. 11/084,738 filed Mar. 16, 2005 and 11/241,749 filed Sep. 29, 2005, the entire contents of which are incorporated herein by reference for all purposes.

### BACKGROUND OF THE INVENTION

Embodiments of the present invention relate to the field of medical devices, and more particularly to a system and method for locally delivering fluids or agents within the body of a patient. Still more particularly, it relates to a system and method for locally delivering fluids or agents into branch blood vessels or body lumens from a main vessel or lumen, respectively, and in particular into renal arteries extending from an aorta in a patient.

Many different medical device systems and methods have been previously described for locally delivering fluids or other agents into various body regions, including body lumens such as vessels, or other body spaces such as organs or heart chambers. Local “fluid” delivery systems may include drugs or other agents, or may even include locally delivering the body's own fluids, such as artificially enhanced blood transport (e.g. either entirely within the body such as directing or shunting blood from one place to another, or in extracorporeal modes such as via external blood pumps etc.). Local “agent” delivery systems are herein generally intended to relate to introduction of a foreign composition as an agent into the body, which may include drug or other useful or active agent, and may be in a fluid form or other form such as gels, solids, powders, gases, etc. It is to be understood that reference to only one of the terms fluid, drug, or agent with respect to local delivery descriptions may be made variously in this disclosure for illustrative purposes, but is not generally intended to be exclusive or omissive of the others; they are to be considered interchangeable where appropriate according to one of ordinary skill unless specifically described to be otherwise.

In general, local agent delivery systems and methods are often used for the benefit of achieving relatively high, localized concentrations of agent where injected within the body in order to maximize the intended effects there and while minimizing unintended peripheral effects of the agent elsewhere in the body. Where a particular dose of a locally delivered agent may be efficacious for an intended local effect, the same dose systemically delivered would be substantially diluted throughout the body before reaching the same location. The agent's intended local effect is equally diluted and efficacy is compromised. Thus systemic agent delivery requires higher dosing to achieve the required localized dose for efficacy, often resulting in compromised safety due to for example systemic reactions or side effects of the agent as it is delivered and processed elsewhere throughout the body other than at the intended target.

Various diagnostic systems and procedures have been developed using local delivery of dye (e.g. radiopaque “contrast” agent) or other diagnostic agents, wherein an external monitoring system is able to gather important physiological information based upon the diagnostic agent's movement or assimilation in the body at the location of delivery and/or at other locations affected by the delivery site. Angiography is one such practice using a hollow, tubular angiography catheter for locally injecting radiopaque dye into a blood chamber or vessel, such as for example coronary arteries in the case of coronary angiography, or in a ventricle in the case of cardiac ventriculography.

Other systems and methods have been disclosed for locally delivering therapeutic agent into a particular body tissue within a patient via a body lumen. For example, angiographic catheters of the type just described above, and other similar tubular delivery catheters, have also been disclosed for use in locally injecting treatment agents through their delivery lumens into such body spaces within the body. More detailed examples of this type include local delivery of thrombolytic drugs such as TPA™, heparin, cumadin, or urokinase into areas of existing clot or thrombogenic implants or vascular injury. In addition, various balloon catheter systems have also been disclosed for local administration of therapeutic agents into target body lumens or spaces, and in particular associated with blood vessels. More specific previously disclosed of this type include balloons with porous or perforated walls that elute drug agents through the balloon wall and into surrounding tissue such as blood vessel walls. Yet further examples for localized delivery of therapeutic agents include various multiple balloon catheters that have spaced balloons that are inflated to engage a lumen or vessel wall in order to isolate the intermediate catheter region from in-flow or out-flow across the balloons. According to these examples, a fluid agent delivery system is often coupled to this intermediate region in order to fill the region with agent such as drug that provides an intended effect at the isolated region between the balloons.

The diagnosis or treatment of many different types of medical conditions associated with various different systems, organs, and tissues, may also benefit from the ability to locally deliver fluids or agents in a controlled manner. In particular, various conditions related to the renal system would benefit a great deal from an ability to locally deliver of therapeutic, prophylactic, or diagnostic agents into the renal arteries.

Acute renal failure (“ARF”) is an abrupt decrease in the kidney's ability to excrete waste from a patient's blood. This change in kidney function may be attributable to many causes. A traumatic event, such as hemorrhage, gastrointestinal fluid loss, or renal fluid loss without proper fluid replacement may cause the patient to go into ARF. Patients may also become vulnerable to ARF after receiving anesthesia, surgery, or a-adrenergic agonists because of related systemic or renal vasoconstriction. Additionally, systemic vasodilation caused by anaphylaxis, and anti-hypertensive drugs, sepsis or drug overdose may also cause ARF because the body's natural defense is to shut down, i.e., vasoconstriction of non-essential organs such as the kidneys. Reduced cardiac output caused by cardiogenic shock, congestive heart failure, pericardial tamponade or massive pulmonary embolism creates an excess of fluid in the body, which can exacerbate congestive heart failure. For example, a reduction in blood flow and blood pressure in the kidneys due to reduced cardiac output can in turn result in the retention of excess fluid in the patient's body, leading, for example, to pulmonary and systemic edema.

Previously known methods of treating ARF, or of treating acute renal insufficiency associated with congestive heart failure (“CHF”), involve administering drugs. Examples of such drugs that have been used for this purpose include, without limitation: vasodilators, including for example papavarine, fenoldopam mesylate, calcium channel blockers, atrial natriuretic peptide (ANP), acetylcholine, nifedipine, nitroglycerine, nitroprusside, adenosine, dopamine, and theophylline; antioxidants, such as for example acetylcysteine; and diuretics, such as for example mannitol, or furosemide. However, many of these drugs, when administered in systemic doses, have undesirable side effects. Additionally, many of these drugs would not be helpful in treating other causes of ARF. For example, a septic shock patient with profound systemic vasodilation often has concomitant severe renal vasoconstriction, administering vasodilators to dilate the renal artery to a patient suffering from systemic vasodilation would compound the vasodilation system wide. In addition, for patients with severe CHF (e.g., those awaiting heart transplant), mechanical methods, such as hemodialysis or left ventricular assist devices, may be implemented. Surgical device interventions, such as hemodialysis, however, generally have not been observed to be highly efficacious for long-term management of CHF. Such interventions would also not be appropriate for many patients with strong hearts suffering from ARF.

The renal system in many patients may also suffer from a particular fragility, or otherwise general exposure, to potentially harmful effects of other medical device interventions. For example, the kidneys as one of the body's main blood filtering tools may suffer damage from exposed to high-density radiopaque contrast dye, such as during coronary, cardiac, or neuro angiography procedures. One particularly harmful condition known as “radiocontrast nephropathy” or “RCN” is often observed during such procedures, wherein an acute impairment of renal function follows exposure to such radiographic contrast materials, typically resulting in a rise in serum creatinine levels of more than 25% above baseline, or an absolute rise of 0.5 mg/dl within 48 hours. Therefore, in addition to CHF, renal damage associated with RCN is also a frequently observed cause of ARF. In addition, the kidneys' function is directly related to cardiac output and related blood pressure into the renal system. These physiological parameters, as in the case of CHF, may also be significantly compromised during a surgical intervention such as an angioplasty, coronary artery bypass, valve repair or replacement, or other cardiac interventional procedure. Therefore, the various drugs used to treat patients experiencing ARF associated with other conditions such as CHF have also been used to treat patients afflicted with ARF as a result of RCN. Such drugs would also provide substantial benefit for treating or preventing ARF associated with acutely compromised hemodynamics to the renal system, such as during surgical interventions.

There would be great advantage therefore from an ability to locally deliver such drugs into the renal arteries, in particular when delivered contemporaneous with surgical interventions, and in particular contemporaneous with radiocontrast dye delivery. However, many such procedures are done with medical device systems, such as using guiding catheters or angiography catheters having outer dimensions typically ranging between about 4 French to about 12 French, and ranging generally between about 6 French to about 8 French in the case of guide catheter systems for delivering angioplasty or stent devices into the coronary or neurovascular arteries (e.g. carotid arteries). These devices also are most typically delivered to their respective locations for use (e.g. coronary ostia) via a percutaneous, translumenal access in the femoral arteries and retrograde delivery upstream along the aorta past the region of the renal artery ostia. A Seldinger access technique to the femoral artery involves relatively controlled dilation of a puncture hole to minimize the size of the intruding window through the artery wall, and is a preferred method where the profiles of such delivery systems are sufficiently small. Otherwise, for larger systems a “cut-down” technique is used involving a larger, surgically made access window through the artery wall.

Accordingly, a local renal agent delivery system for contemporaneous use with other retrogradedly delivered medical device systems, such as of the types just described above, would preferably be adapted to allow for such interventional device systems, in particular of the types and dimensions just described, to pass upstream across the renal artery ostia (a) while the agent is being locally delivered into the renal arteries, and (b) while allowing blood to flow downstream across the renal artery ostia, and (c) in an overall cooperating system that allows for Seldinger femoral artery access. Each one of these features (a), (b), or (c), or any sub-combination thereof, would provide significant value to patient treatment; a local renal delivery system providing for the combination of all three features is so much the more valuable.

Notwithstanding the clear needs for and benefits that would be gained from such local drug delivery into the renal system, the ability to do so presents unique challenges as follows.

In one regard, the renal arteries extend from respective ostia along the abdominal aorta that are significantly spaced apart from each other circumferentially around the relatively very large aorta. Often, these renal artery ostia are also spaced from each other longitudinally along the aorta with relative superior and inferior locations. This presents a unique challenge to locally deliver drugs or other agents into the renal system on the whole, which requires both kidneys to be fed through these separate respective arteries via their uniquely positioned and substantially spaced apart ostia. This becomes particularly important where both kidneys may be equally at risk, or are equally compromised, during an invasive upstream procedure—or, of course, for any other indication where both kidneys require local drug delivery. Thus, an appropriate local renal delivery system for such indications would preferably be adapted to feed multiple renal arteries perfusing both kidneys.

In another regard, mere local delivery of an agent into the natural, physiologic blood flow path of the aorta upstream of the kidneys may provide some beneficial, localized renal delivery versus other systemic delivery methods, but various undesirable results still arise. In particular, the high flow aorta immediately washes much of the delivered agent beyond the intended renal artery ostia. This reduces the amount of agent actually perfusing the renal arteries with reduced efficacy, and thus also produces unwanted loss of the agent into other organs and tissues in the systemic circulation (with highest concentrations directly flowing into downstream circulation).

In still a further regard, various known types of tubular local delivery catheters, such as angiographic catheters, other “end-hole” catheters, or otherwise, may be positioned with their distal agent perfusion ports located within the renal arteries themselves for delivering agents there, such as via a percutaneous translumenal procedure via the femoral arteries (or from other access points such as brachial arteries, etc.). However, such a technique may also provide less than completely desirable results.

For example, such seating of the delivery catheter distal tip within a renal artery may be difficult to achieve from within the large diameter/high flow aorta, and may produce harmful intimal injury within the artery. Also, where multiple kidneys must be infused with agent, multiple renal arteries must be cannulated, either sequentially with a single delivery device, or simultaneously with multiple devices. This can become unnecessarily complicated and time consuming and further compound the risk of unwanted injury from the required catheter manipulation. Moreover, multiple dye injections may be required in order to locate the renal ostia for such catheter positioning, increasing the risks associated with contrast agents on kidney function (e.g. RCN)—the very organ system to be protected by the agent delivery system in the first place. Still further, the renal arteries themselves, possibly including their ostia, may have pre-existing conditions that either prevent the ability to provide the required catheter seating, or that increase the risks associated with such mechanical intrusion. For example, the artery wall may be diseased or stenotic, such as due to atherosclerotic plaque, clot, dissection, or other injury or condition. Finally, among other additional considerations, previous disclosures have yet to describe an efficacious and safe system and method for positioning these types of local agent delivery devices at the renal arteries through a common introducer or guide sheath shared with additional medical devices used for upstream interventions, such as angiography or guide catheters. In particular, to do so concurrently with multiple delivery catheters for simultaneous infusion of multiple renal arteries would further require a guide sheath of such significant dimensions that the preferred Seldinger vascular access technique would likely not be available, instead requiring the less desirable “cut-down” technique.

In addition to the various needs for locally delivering agents into branch arteries described above, much benefit may also be gained from simply locally enhancing blood perfusion into such branches, such as by increasing the blood pressure at their ostia. In particular, such enhancement would improve a number of medical conditions related to insufficient physiological perfusion into branch vessels, and in particular from an aorta and into its branch vessels such as the renal arteries.

Certain other approaches have provided surgical device assemblies and methods intended to enhance blood delivery into branch arteries extending from an aorta. For example, intra-aortic balloon pumps (IABPs) have been disclosed for use in diverting blood flow into certain branch arteries. One such technique involves placing an IABP in the abdominal aorta so that the balloon is situated slightly below (proximal to) the branch arteries. The balloon is selectively inflated and deflated in a counterpulsation mode (by reference to the physiologic pressure cycle) so that increased pressure distal to the balloon directs a greater portion of blood flow into principally the branch arteries in the region of their ostia. However, the flow to lower extremities downstream from such balloon system can be severely occluded during portions of this counterpulsing cycle. Moreover, such previously disclosed systems generally lack the ability to deliver drug or agent to the branch arteries while allowing continuous and substantial downstream perfusion sufficient to prevent unwanted ischemia.

It is further noted that, despite the renal risks described in relation to radiocontrast dye delivery, and in particular RCN, in certain circumstances local delivery of such dye or other diagnostic agents is indicated specifically for diagnosing the renal arteries themselves. For example, diagnosis and treatment of renal stenosis, such as due to atherosclerosis or dissection, may require dye injection into a subject renal artery. In such circumstances, enhancing the localization of the dye into the renal arteries may also be desirable. In one regard, without such localization larger volumes of dye may be required, and the dye lost into the downstream aortic flow may still be additive to impacting the kidney(s) as it circulates back there through the system. In another regard, an ability to locally deliver such dye into the renal artery from within the artery itself, such as by seating an angiography catheter there, may also be hindered by the same stenotic condition requiring the dye injection in the first place (as introduced above). Still further, patients may have stent-grafts that may prevent delivery catheter seating.

Notwithstanding the interest and advances toward locally delivering agents for treatment or diagnosis of organs or tissues, the previously disclosed systems and methods summarized immediately above generally lack the ability to effectively deliver agents from within a main artery and locally into substantially only branch arteries extending therefrom while allowing the passage of substantial blood flow and/or other medical devices through the main artery past the branches. This is in particular the case with previously disclosed renal treatment and diagnostic devices and methods, which do not adequately provide for local delivery of agents into the renal system from a location within the aorta while allowing substantial blood flow continuously downstream past the renal ostia and/or while allowing distal medical device assemblies to be passed retrogradedly across the renal ostia for upstream use. Much benefit would be gained if agents, such as protective or therapeutic drugs or radiopaque contrast dye, could be delivered to one or both of the renal arteries in such a manner.

Several more recent advances have included local flow assemblies using tubular members of varied diameters that divide flow within an aorta adjacent to renal artery ostia into outer and inner flow paths substantially perfusing the renal artery ostia and downstream circulation, respectively. Such advances further include delivering fluid agent primarily into the outer flow path for substantially localized delivery into the renal artery ostia. These systems and methods represent exciting new developments toward localized diagnosis and treatment of pre-existing conditions associated with branch vessels from main vessels in general, and with respect to renal arteries extending from abdominal aortas in particular.

However, while these approaches in one regard provide benefit by removing the need to cannulate each renal artery of the bi-lateral renal system, substantial benefit would still be gained conversely from a device system and method that allows for direct bi-lateral renal artery infusion without the need to deploy flow diverters or isolators into the high-flow abdominal aorta. In one particular example, patients that suffer from abdominal aortic aneurysms may not be suitable for standard delivery systems with flow diverters or isolators that are sized for normal arteries. In another regard, direct renal artery infusion allows for reduced occlusion to downstream aortic blood flow, or conversely more downstream flow may be preserved. Still further, the ability to truly isolate drug to only the renal system, without the potential for downstream leaking or loss into the systemic circulation, may be maximized.

A need therefore still exists for improved devices and methods for locally delivering agents bi-laterally into each of two renal arteries perfusing both kidneys of a patient while a substantial portion of aortic blood flow is allowed to perfuse downstream across the location of the renal artery ostia and into the patient's lower extremities.

A need still exists for improved devices and methods for efficiently gaining percutaneous translumenal access into each side of the kidney system via their separate renal artery ostia along the abdominal aortic wall, so that procedures such as fluid agent delivery may be performed locally within both sides of the renal system.

A need still exists for improved devices and methods for locally delivering fluid agents into a renal artery from a location within the aorta of a patient adjacent the renal artery's ostium along the aorta wall.

A need still exists for improved devices and methods for locally isolating delivery of fluids or agents into the renal arteries of a patient, and while allowing other treatment or diagnostic devices and systems, such as angiographic or guiding catheter devices and related systems, to be delivered across the location.

A need still exists for improved devices and methods for locally delivering fluids or agents into the renal arteries of a patient, for prophylaxis or diagnostic procedures related to the kidneys.

A need still exists for improved devices and methods for locally isolating delivery of fluids or agents into the renal arteries of a patient in order to treat, protect, or diagnose the renal system adjunctive to performing other contemporaneous medical procedures such as angiograms other translumenal procedures upstream of the renal artery ostia.

A need still exists for improved devices and methods for delivering both a local renal drug delivery system and at least one adjunctive distal interventional device, such as an angiographic or guiding catheter, through a common delivery sheath.

A need also still exists for improved devices and methods for delivering both a local renal drug delivery system and at least one adjunctive distal interventional device, such as an angiographic or guiding catheter, through a single access site, such as a single femoral arterial puncture.

A need also still exists for improved devices and methods for treating, and in particular preventing, ARF, and in particular relation to RCN or CHF, by locally delivering renal protective or ameliorative drugs into the renal arteries, such as contemporaneous with radiocontrast injections such as during angiography procedures.

In addition to these particular needs for selective fluid delivery into a patient's renal arteries via their ostia along the aorta, other similar needs also exist for locally isolated fluid delivery into other branch vessels or lumens extending from other main vessels or lumens, respectively, in a patient.

### BRIEF SUMMARY OF THE INVENTION

The present invention provides treatment delivery systems that facilitate a single stick entry for renal infusion during peripheral vascular interventional procedures. Improved introducer sheaths or main outer delivery sheaths channel passage of both renal and peripheral catheter systems. Relatedly, embodiments provide introducer sheaths having exit port designs that allow passage of catheters and other treatment devices. Improved back, proximal, or workspace ends of treatment systems are also provided.

In a first aspect, embodiments of the present invention provide a system for delivering treatment to a renal artery and a peripheral artery. The system includes a renal catheter, a peripheral catheter, and an introducer sheath having a first lumen and a second lumen. The first lumen can be configured to receive the renal catheter and can be sized to extend from a patient insertion site to a femoral or iliac artery location near or distal to a patient aortic branch. The second lumen can be configured to receive the peripheral catheter and can be sized to extend from the patient insertion site to an opposite femoral or iliac artery location near or distal to the patient aortic branch. In some cases, at least one of the first lumen and the second lumen includes a variable cross-section. The introducer sheath may include an exit port in communication with the first lumen. The introducer sheath may also include a proximal section, a distal section, a tapered section therebetween having an exit port. In some cases, the introducer sheath includes a coil disposed at the proximal section, the distal section, and the tapered section. The coil can have a first pitch at the proximal and distal sections and a second pitch at the tapered section. In some cases, the exit port includes a plug having a slit. In some cases, the system includes a drug infusion source in operative association with the renal catheter.

In another aspect, embodiments of the present invention provide a system for delivering treatment to a renal artery and a peripheral artery. The system can include a renal catheter having a distal bifurcation, a peripheral catheter, and an introducer sheath having a first lumen and a second lumen. The first lumen can be configured to receive the renal catheter and can be separated from the second lumen by a flexible separator flap. The second lumen can be configured to receive the peripheral catheter. In some cases, at least one of the first lumen and the second lumen includes a variable cross-section. The introducer sheath may include an exit port in communication with the first lumen. The introducer sheath may also include a proximal section, a distal section, and a tapered section therebetween having an exit port. The introducer sheath may include a coil disposed at the proximal section, the distal section, and the tapered section. The coil can have a first pitch at the proximal and distal sections and a second pitch at the tapered section. In some cases, the system includes a drug source in operative association with the renal catheter.

In another aspect, embodiments of the present invention provide a method of delivering treatment to a renal artery and a peripheral artery. The method can include positioning an introducer sheath in an iliac artery, advancing a renal catheter through a first lumen of the introducer sheath to a location at or near the renal artery, and advancing a peripheral catheter through a second lumen of the introducer sheath to a location at or near the peripheral artery. The first lumen and the second lumen of the introducer sheath can be separated by a flexible separator flap. In some cases, at least one of the first lumen and the second lumen includes a variable cross-section. The method may also include advancing the renal catheter through an exit port of the introducer sheath, where the exit port is in communication with the first lumen. In some cases, the method also includes providing a renal treatment via the renal catheter, and providing a peripheral treatment via the peripheral catheter. Relatedly, the method may include delivering a renal therapeutic agent via the renal catheter, where the renal therapeutic agent improves kidney function, prevents kidney damage, or both.

In still another aspect, embodiments of the present invention provide a system for delivering treatment to a renal artery and a peripheral artery. The system can include, for example, a renal catheter, a peripheral catheter, and an introducer sheath having an exit port. The introducer sheath can be configured to receive the renal catheter and the peripheral catheter and can be sized to extend from a patient insertion site past a femoral or iliac artery location near or distal to a patient aortic branch to an opposite femoral or iliac artery location near or distal to the patient aortic branch, such that when the introducer sheath is placed in the patient, the exit port is situated at or near the a femoral or iliac artery location near or distal to the patient aortic branch. In some cases, the introducer sheath includes a proximal section, a distal section, and a tapered section therebetween. The exit port may be disposed at the tapered section. The introducer sheath may also include a coil disposed at the proximal section, the distal section, and the tapered section. The coil can have a first pitch at the proximal and distal sections and a second pitch at the tapered section. Optionally, the renal catheter may include a bifurcated distal end.

In still another aspect, embodiments of the present invention include a method of positioning a renal treatment system and a peripheral treatment system. The method may include positioning an introducer sheath in an iliac artery, advancing a renal catheter of the renal treatment system through a lumen of the introducer sheath to a location at or near a renal artery, and advancing a peripheral catheter of the peripheral treatment system through the lumen of the introducer sheath to a location at or near a peripheral artery. The method may also include advancing the renal catheter through an exit port of the introducer sheath. The exit port can be located proximal to a distal end of the introducer sheath. In some cases, the method includes providing a renal treatment via the renal catheter, and providing a peripheral treatment via the peripheral catheter. The method can include delivering a renal therapeutic agent via the renal catheter, where the renal therapeutic agent improves kidney function, prevents kidney damage, or both.

In another aspect, embodiments of the present invention provide a system for delivering treatment to a renal artery and a peripheral artery. The system can include, for example, a plurality of renal catheters, a plurality of peripheral catheters, and an introducer sheath having a plurality of renal catheter lumens and a plurality of peripheral catheter lumens. Each of the plurality of renal catheter lumens can be configured to receive at least one of the plurality of renal catheters and can be sized to extend from a patient insertion site to a femoral or iliac artery location near or distal to a patient aortic branch. Each of the plurality of peripheral catheter lumens can be sized to extend from the patient insertion site to an opposite femoral or iliac artery location near or distal to the patient aortic branch. In some cases, each of the plurality of renal catheter lumens and peripheral catheter lumens exit the introducer sheath at distinct locations along a length of the sheath. In some cases, at least one lumen of the plurality of renal catheter lumens and the plurality of peripheral catheter lumens comprises a variable cross-section.

In yet another aspect, embodiments of the present invention provide a system for delivering treatment to a renal artery and a peripheral artery. The system includes a plurality of renal catheters, a plurality of peripheral catheters, and an introducer sheath having a plurality of renal catheter lumens and a plurality of peripheral catheter lumens. At least one of the renal catheters may include a distal bifurcation. Each of the plurality of renal catheter lumens can be configured to receive at least one of the plurality of renal catheters. Each of the plurality of peripheral catheter lumens can be configured to receive at least one of the plurality of peripheral catheters. In some cases, at least one of the renal catheter lumens is separated from at least one of the peripheral catheter lumens by a flexible separator flap. Each of the plurality of renal catheter lumens and peripheral catheter lumens may exit the introducer sheath at distinct locations along a length of the sheath. In some cases, at least one lumen of the plurality of renal catheter lumens and the plurality of peripheral catheter lumens includes a variable cross-section.

In another aspect, embodiments of the present invention include a method of delivering treatment to a renal artery and a peripheral artery. The method may include, for example, positioning an introducer sheath in an iliac artery, where the introducer sheath includes a plurality of renal catheter lumens and a plurality of peripheral catheter lumens. The method may also include advancing each of a plurality of renal catheters through a respective renal catheter lumen of the plurality of renal catheter lumens to a location at or near the renal artery. Further, the method may include advancing each of a plurality of peripheral catheters through a respective peripheral catheter lumen of the plurality of peripheral catheter lumens to a location at or near the peripheral artery. In some cases, at least one of the renal catheter lumens is separated from at least one of the peripheral catheter lumens by a flexible separator flap. Each of the plurality of renal catheter lumens and peripheral catheter lumens may exit the introducer sheath at distinct locations along a length of the sheath. In some cases, at least one lumen of the plurality of renal catheter lumens and the plurality of peripheral catheter lumens comprises a variable cross-section. Optionally, the method may include providing a renal treatment via at least one of the plurality of renal catheters, providing a peripheral treatment via at least one of the plurality of peripheral catheters, or both. Relatedly, the method may include delivering a renal therapeutic agent via at least one of the plurality of renal catheters. The renal therapeutic agent may improve kidney function, prevent kidney damage, or both.

### BRIEF DESCRIPTION OF THE DRAWINGS

FIGS. 1A-1C illustrate a system for delivering a renal treatment via a renal artery and a peripheral treatment via a peripheral artery according to embodiments of the present invention.

FIG. 2 illustrates a system for delivering a renal treatment via a renal artery and a peripheral treatment via a peripheral artery according to embodiments of the present invention.

FIG. 3 depicts an introducer sheath according to embodiments of the present invention.

FIG. 4 depicts an introducer sheath according to embodiments of the present invention.

FIG. 5 depicts an introducer sheath according to embodiments of the present invention.

FIG. 6 depicts an introducer sheath according to embodiments of the present invention.

FIG. 7 depicts an introducer sheath according to embodiments of the present invention.

FIG. 8 depicts a renal catheter according to embodiments of the present invention.

FIG. 9 depicts a Y-hub assembly according to embodiments of the present invention.

FIG. 10 depicts a treatment sheath according to embodiments of the present invention.

FIG. 11 depicts a treatment sheath according to embodiments of the present invention.

FIG. 12 depicts a treatment sheath according to embodiments of the present invention.

FIG. 13 illustrates a system for delivering a renal treatment via a renal artery and a peripheral treatment via a peripheral artery according to embodiments of the present invention.

FIG. 14 illustrates a system for delivering a renal treatment via a renal artery and a peripheral treatment via a peripheral artery according to embodiments of the present invention.

FIGS. 15-15C depict an introducer sheath according to embodiments of the present invention.

FIGS. 16A-16C illustrates an exemplary method of using system for delivering a renal treatment via a renal artery and a peripheral treatment via a peripheral artery according to embodiments of the present invention.

### DETAILED DESCRIPTION OF THE INVENTION

In some embodiments, a treatment sheath can be delivered over a guidewire through an introducer or main outer sheath to a site of renal artery cannulation. Once the guidewire is removed, the treatment catheter can be delivered through the treatment sheath. Upon delivery and deployment of the catheter branches, the treatment sheath can be retracted from the body to allow space for passage of the device used in peripheral intervention.

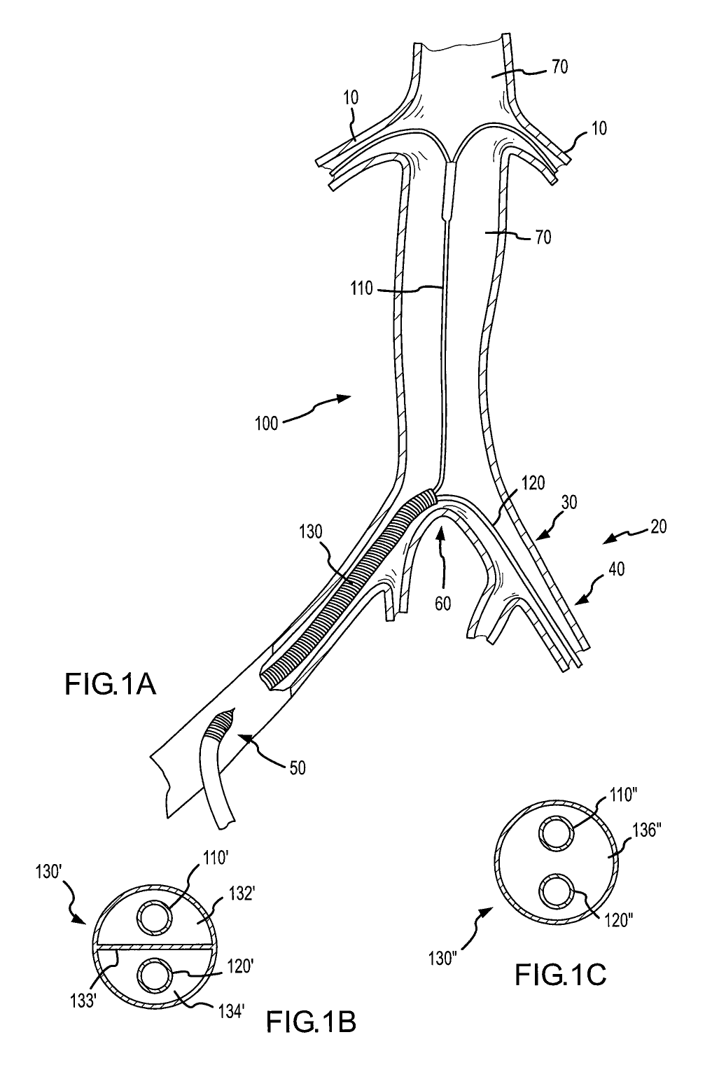

Turning now to the drawings, FIG. 1A shows a system 100 for delivering treatment to a renal artery 10 and a peripheral artery 20 of a patient according to embodiments of the present invention. A peripheral artery may encompass, for example, an iliac artery 30, a femoral artery 40, and the like. System 100 includes a renal treatment system having a renal catheter 110, a peripheral treatment system having a peripheral catheter 120, and an introducer sheath 130. In some cases, a renal treatment system includes a renal treatment sheath. Similarly, a peripheral treatment system can include a peripheral treatment sheath. In this embodiment, introducer sheath extends from an insertion point 50 to an aortic bifurcation 60. Renal catheter 110 and peripheral catheter 120 can each exit introducer sheath 130 laterally and navigate toward their own respective delivery site For example, renal catheter 110 can exit introducer sheath 130 and travel through aorta 70 to the renal arteries 10. As shown in FIG. 1B, in some embodiments an introducer sheath 130′ can have a first lumen 132′ configured to receive a renal catheter 110′ and a second lumen 134′, and a second lumen 134′ configured to receive a peripheral catheter 120′. First lumen 132′ and second lumen 134′ can be divided by a separator 133′. In some aspects, a separator may include a flexible separator flap. FIG. 1C depicts an introducer sheath 130″ having a single lumen 136″ configured to receive a renal catheter 110″ and a peripheral catheter 120″. In some cases, an introducer sheath can be sized to extend from an insertion point to an opening in the iliac artery. In related cases, an introducer sheath can be sized to extend from an insertion point to an aortic bifurcation.

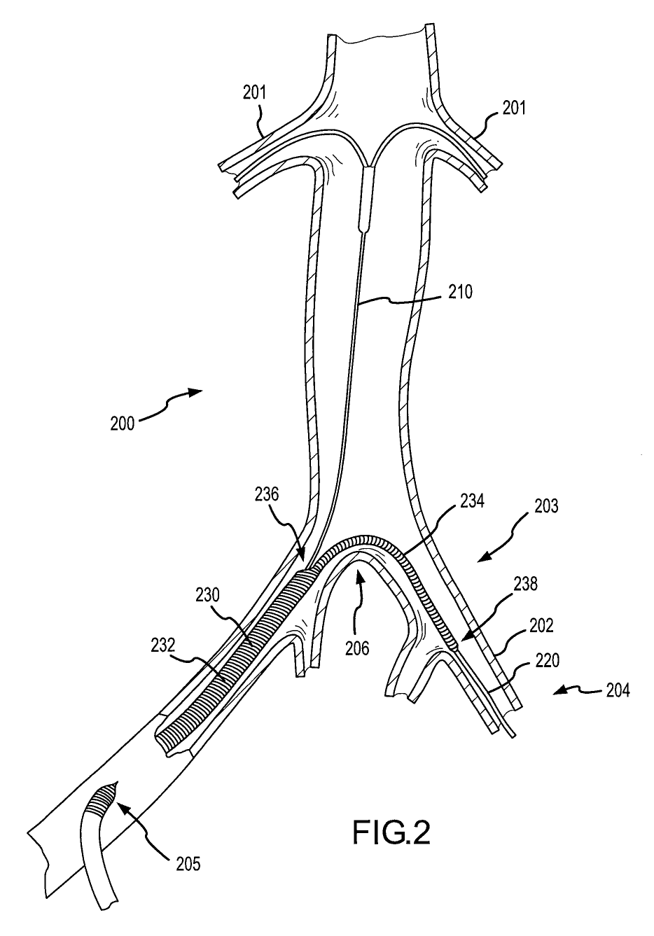

An exemplary system 200 for delivering treatment to a renal artery 201 and a peripheral artery 202 is illustrated in FIG. 2. System 200 includes a renal catheter 210, a peripheral catheter 220, and an introducer sheath 230. Introducer sheath extends from an insertion point 205 across an aortic bifurcation 206 to a peripheral artery 202, which may be an iliac artery 203 or a femoral artery 204. Renal catheter 210 and peripheral catheter 220 can each exit introducer sheath 230 laterally and navigate toward their own respective delivery site. Introducer sheath 230 includes a first portion 232 configured to receive renal catheter 210 and peripheral catheter 220, and a second portion 234 configured to receive peripheral catheter 220. Introducer sheath also includes an exit port 236 through which renal catheter 210 can pass. Often, first portion 232 has a larger diameter than second portion 234. In some embodiments, renal catheter 210 has an outer diameter of about 1.5 F and peripheral intervention system has an outer diameter or equivalent thereof of about 8 F. Introducer sheath 230 passes through a first iliac artery and extends to curve toward the opposite iliac artery, which can facilitate a convenient navigation of the peripheral system. Exit port 236 may be disposed anywhere along the length of introducer sheath 230. As shown in this embodiment, exit port 236 is disposed at a location proximal to a distal exit 238 of sheath 230. Exit port 236 allows renal catheter 210 to exit introducer sheath 230 at or near aortic bifurcation 206.

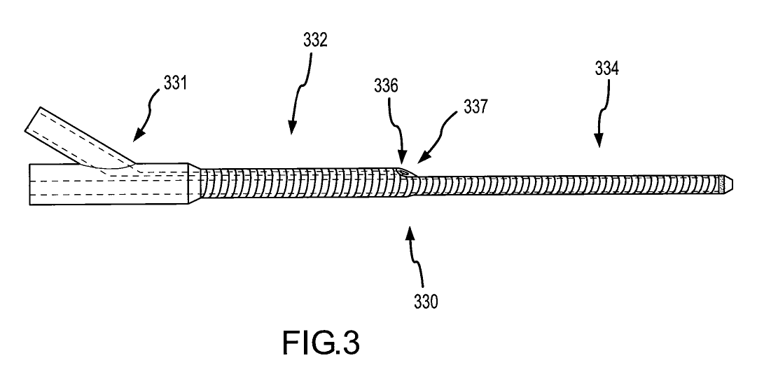

FIG. 3 depicts an introducer sheath 330 having a proximal portion 332 and a distal portion 334. Proximal portion is coupled with a Y-hub 331. In some embodiments, proximal portion 332 can have an inner diameter within a range from about 9.5 F to about 10 F, and distal portion 334 can have an inner diameter within a range from about 8 F to about 8.5 F. In related embodiments, proximal portion 332 can have an inner diameter within a range from about 7.5 F to about 12 F, and distal portion 334 can have an inner diameter within a range from about 5.5 F to about 9.5 F. Optionally, proximal portion 332 can have an inner diameter of about 8 F, and distal portion 334 can have an inner diameter of about 7 F. Introducer sheath 330 also includes an exit port 336 disposed at a transition portion 337 situated between proximal portion 332 and distal portion 334. Exit port 336 can include an open aperture sized to receive elements of a treatment system, such as a catheter or a sheath.

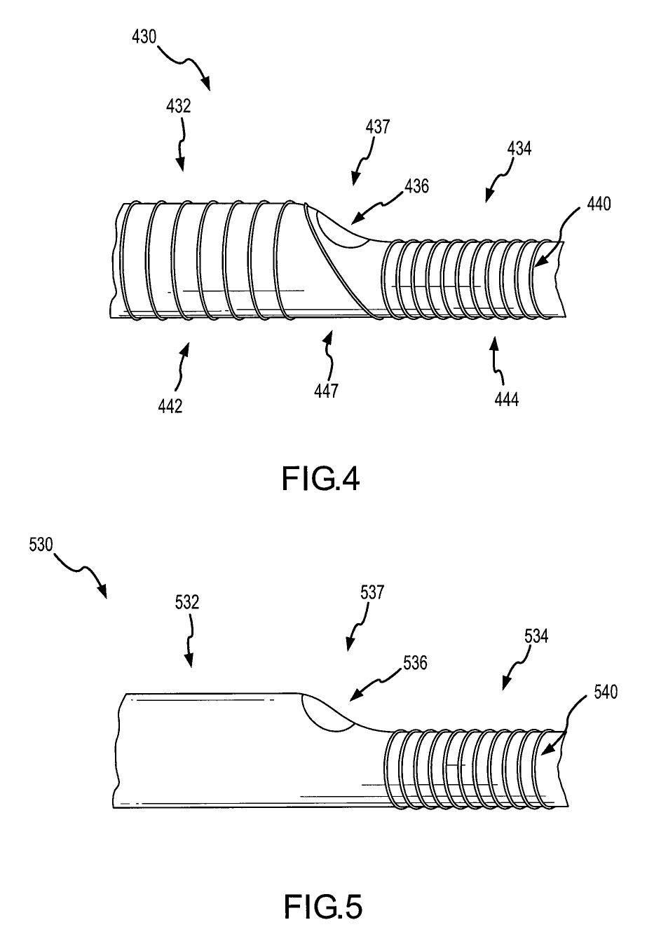

As shown in FIG. 4, an introducer sheath 430 may include a coiling or coil 440. In some embodiments, a coil provides structural reinforcement to an introducer sheath. Introducer sheath 430 includes a first proximal portion 432, a second distal portion 434, and a transition portion 437 disposed therebetween. Transition portion 437 often defines a tapered shape or profile and includes an exit port 436. In a corresponding fashion, coil 440 includes a first portion 442, a second portion 444, and a transition portion 447 disposed therebetween. In some embodiments, coil 440 is wrapped at a first pitch at first portion 442 and second portion 444, and is wrapped at a second pitch at transition portion 447. The second pitch at transition portion 447 can be spaced sufficiently to allow enough space for exit port 436 to provide an aperture or opening in introducer sheath 430 without piercing the coil or traversing the path of the coil. Accordingly, the exit port can be created without disturbing the continuity of the coil. In some cases, the transition portion 447 reflects a jump in the coil pitch for one full wrap. Coils may be disposed on an outside layer of the introducer sheath, on an inside layer of the introducer sheath, or on an intermediate layer of the introducer sheath.

In some embodiments, an introducer sheath may include a coil layer disposed only distal to the transition portion. FIG. 5 illustrates an introducer sheath 530 having a first portion 532, a second portion 534, and a tapered transition portion 537 disposed therebetween. Transition portion 537 may present an exit port 536. Introducer sheath 530 also includes a coil 540 disposed at or near second portion 534. In some cases, the wall thickness of first portion 532 may be greater than the wall thickness of second portion 534, as compensation for the torquability on the proximal and wider end of the first portion of the introducer sheath. Such configurations can help maintain a uniform or desired torquing profile along the length of introducer sheath 530.

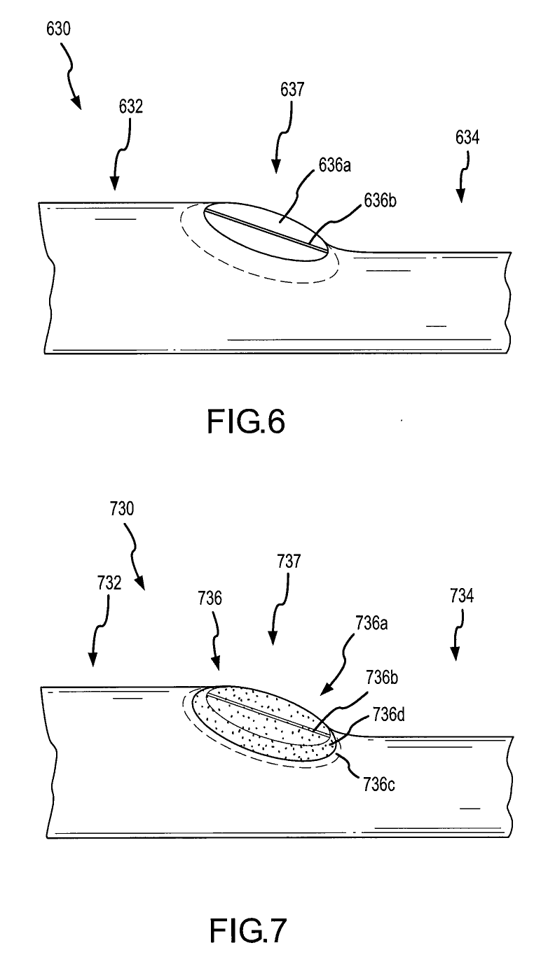

As noted above with reference to FIG. 3, an exit port can have an open aperture through which a renal catheter or treatment sheath can pass. The exit port may or may not include a covering or plug. As shown in FIG. 6, an introducer sheath 630 can include a proximal portion 632, a distal portion 634, and a transition portion 637 disposed therebetween. Transition portion 637 includes an exit port 636 having a plug 636a with a passage slit 636b. In some embodiments, plug 636a includes an elastomeric material such as silicone. Plug 636a can be formed in contour with the overall surface profile of the introducer sheath. Passage slit 636b can be sized to allow passage of elements of a renal treatment system, such as a renal treatment sheath or a renal catheter. Relatedly, FIG. 7 shows an introducer sheath 730 a proximal portion 732, a distal portion 734, and a transition portion 737 disposed therebetween. Transition portion 737 includes an exit port 736 having a plug 736a with a passage slit 736b. Plug 736a can be formed in contour with the overall surface profile of the introducer sheath. Passage slit 736b can be sized to allow passage of a renal catheter. In some embodiments, plug 736a includes one or more layers of elastomeric material. For example, plug 736a may include an inner layer 736c and an outer layer 736d. In some cases, plug 736a includes an embedded inner silicone layer and an outer layer of low-durometer Pebax® covering the entire section circumferentially to aid in supporting the integrity of plug 736a.

Embodiments of the present invention provide various solutions for a functional back-end interface that can facilitate the delivery and retrieval of a renal treatment system, a peripheral treatment system, or both, in an integrated single stick system. FIG. 13 shows a system 1300 for delivering treatment to a renal artery and a peripheral artery. System 1300 includes a renal catheter 1310 and a peripheral catheter 1320. Renal catheter 1310 may include a proximal section 1312 disposed exterior to the patient's body, and a distal section 1314 disposed interior to the patient's body. Proximal section 1312 presents an extended or lengthened workable catheter length. Advantageously, the extended workable length of proximal section 1312 allows for facile treatment sheath removal without disruption of a fluid line. In some embodiments, renal catheter 1310 includes a proximal section 1312 having a 1.5 F NiTi section and a distal section 1314 having a 2-4 F Pebax® section. The diameter of distal section 1314 can be selected so as to provide certain desired characteristics. For example, the distal section diameter can be selected to allow for decreased mechanical resistance to flow and for desired handling characteristics when a clinician or operator rolls or spools the workable length.

Other solutions for a functional back-end interface involve the removal of an treatment sheath from a catheter. Such solutions may or may not include catheters having an extended workable length. FIG. 9 illustrates an exemplary Y-hub 931 and introducer sheath 930 combination, wherein Y-hub is coupled with a proximal end 932 of introducer sheath 930. Y-hub 931 permits the entry of a renal treatment system and a peripheral treatment system therethrough. Y-hub can also act, often simultaneously, to allow infusion of a fluid through a separate sub-port 931a. In some cases, sub-port 931a is in operative association with a Y-hub branch that receives a renal catheter. Optionally, sub-port 931a can be in operative association with a Y-hub branch that receives a peripheral catheter. In some cases, a Y-hub may include such a sub-port on both Y-hub branches. Sub-port 931a can be coupled or in fluid communication with an infusion pump 980, and can enter Y-hub 930 at an infusion insertion section thereof. Infusion insertion section 985 can be disposed or isolated within the Y-hub branch between two plugs 986. In the embodiment shown here, catheter 910 includes perforations or apertures 913 that allow passage of infusion fluid from sub-port 931a into catheter 910. A sub-port can be connected with an infusion pump, and can enter a renal catheter entry lumen of a Y-hub at a section that is isolated by two plugs. The plugs may allow for selective passage of a catheter or treatment sheath, but inhibit passage of fluids under certain pressures. For example, the plugs may allow for the passage of a catheter or treatment sheath, but may prevent passage of a fluid that is pressurized at 20 psi or less. A catheter can include perforations configured for alignment between the two plugs, such that fluid supplied by a infusion pump can pressurize in a sub-chamber or infusion insertion section between the two plugs and enter the catheter through the perforations. Embodiments such as these allow for the infusion of fluid into a catheter while also permitting removal and reentry of a treatment sheath for both delivery and recapture of a treatment catheter and any branches or devices attached thereto.

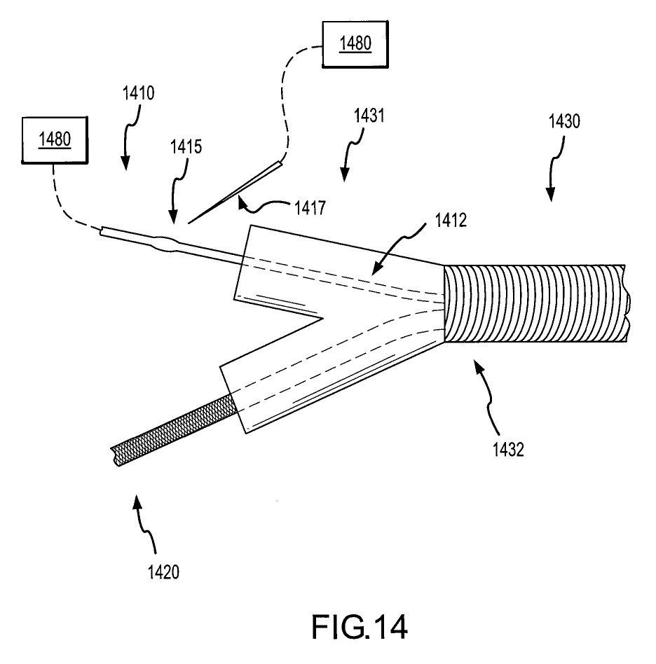

FIG. 14 illustrates an exemplary back-end interface having a Y-hub 1431 in combination with an introducer sheath 1430, wherein Y-hub is coupled with a proximal end 1432 of introducer sheath 1430. Y-hub 1431 permits the entry of a renal treatment system 1410 and a peripheral treatment or intervention system 1420 therethrough. Renal treatment system 1410 may include a needle pierce section 1415 adapted to receive a needle 1417, which may be coupled with an infusion source 1482. Needle pierce section 1415 can be disposed along the renal treatment system at a location where the renal treatment system exits the patient's body. Thus, introducer sheath 1430 can be delivered having an infusion pump 1480 connected or in fluid communication with a proximal end of the renal treatment system or catheter. When removal of treatment sheath 1430 is desired or required, the renal catheter can be disconnected at a proximal end from infusion pump 1480, and infusion can resume or continue from needle pierce section 1415. Renal treatment system 1410 may include a proximal catheter 1412 that includes a NiTi shaft.

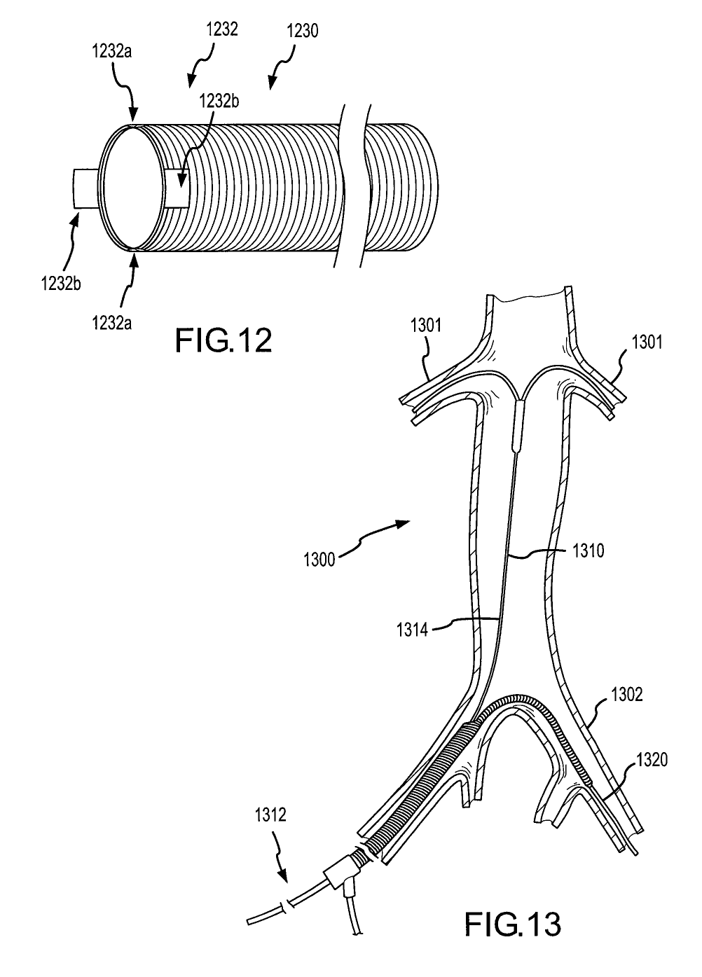

Other back-end interface solutions include treatment sheaths that can be removed from a catheter via a longitudinal dissection configuration. FIG. 12 shows a proximal portion 1232 of a treatment sheath 1230. Proximal portion 1232 may include a pierce groove 1232a and a tear-away grip 1332b. With this peel-away configuration, when a treatment sheath is retrieved it can be easily removed. As desired, a peel-away configuration may or may not be used on conjunction with a catheter having an extended or lengthened workable catheter length. FIG. 10 illustrates an treatment sheath 1030 having a longitudinal clip section 1030a that can be clipped off from and reclipped on to a catheter line. In some cases, longitudinal clip section presents a continuous clip along the length of treatment sheath 1030. FIG. 11 illustrates a treatment sheath 1130 having an array of serially arranged sheath sections 1130a having clips 1130b, where sheath sections 1130a are held in place by a supporting structure such as a wire 1130c.

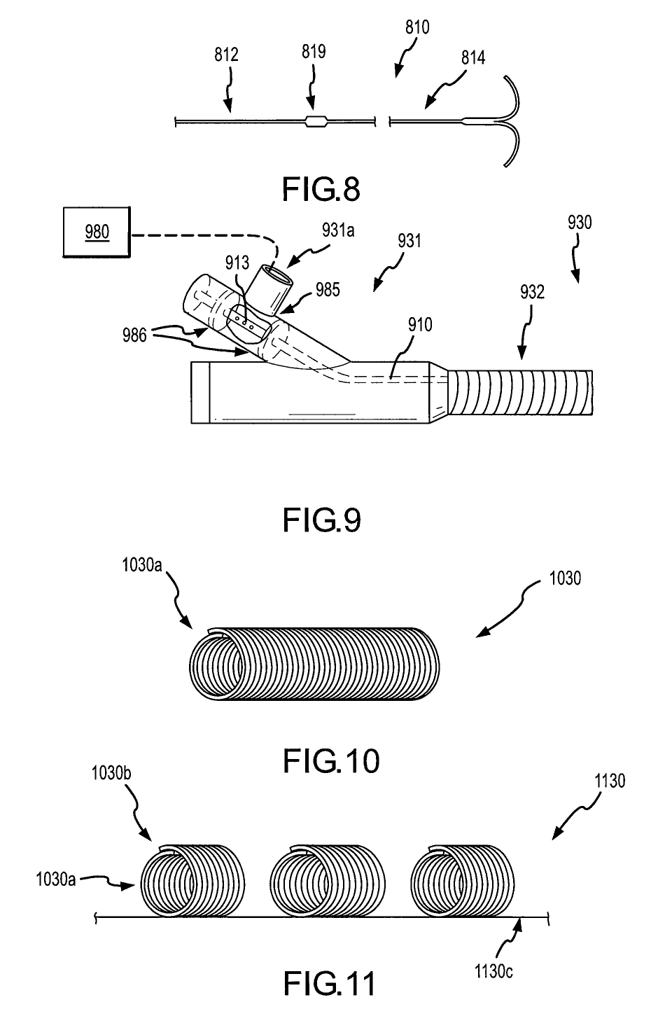

Another back-end interface solution includes a catheter connector. For example, as shown in FIG. 8, renal treatment system 810 includes a catheter connector 819 than can allow disconnection and reconnection of a catheter proximal section 812 and a catheter distal section 814. Such disconnection and reconnection of the catheter line can provide for facile removal of a treatment sheath. As desired, such a configuration may or may not be used on conjunction with a catheter having an extended or lengthened workable catheter length.

A wide variety of variable lumen construction sheaths may be used. In some embodiments, an intervention system may include an introducer sheath with variable lumen construction that has the ability to serve asynchronously or synchronously as a conduit for multiple devices. Many traditional sheaths and catheters are constructed with a single lumen. Multi lumen catheters are also common (such as balloon catheters) and multi-lumen sheaths have likewise been described in, for example, U.S. Pat. No. 4,769,005 and U.S. patent application Ser. No. 11/073,421 filed Mar. 4, 2005, the entire disclosures of which are hereby incorporated by reference for all purposes.

In some embodiments, the present sheath may differ from known sheaths in that it can be designed to handle multiple products through its multiple lumens either simultaneously and/or at different times, and can be adapted with variable luminal cross sections to allow for passage of devices of multiple larger sizes through different lumens at different times during a given procedure.

While it is possible to insert multiple devices through a single-lumen design, a dual lumen or multiple lumen design can provide the advantages of separating the multiple devices to reduce the propensity for tangling and also allows fluid separation between the lumens to allow for more selective infusions if desired.

In some embodiments, the present sheath has multiple lumens with varying points of exit from the sheath designed to allow passage of multiple devices to remote regions of the vasculature simultaneously or at different times during the same procedure. As an example, a sheath can have an outer diameter (OD) similar to that of a standard, commercially available 10F vascular introducer sheath. In a dual lumen construction sheath, wherein the lumens have variable cross-sections, two devices with crossing profiles of about 8 F can pass through either lumen at different times, or, alternatively, a device up to 8 F can pass through one lumen simultaneously with a device of up to about 2 F in the other lumen. Combinations between these extremes are also contemplated (e.g., two 5 F devices simultaneously).

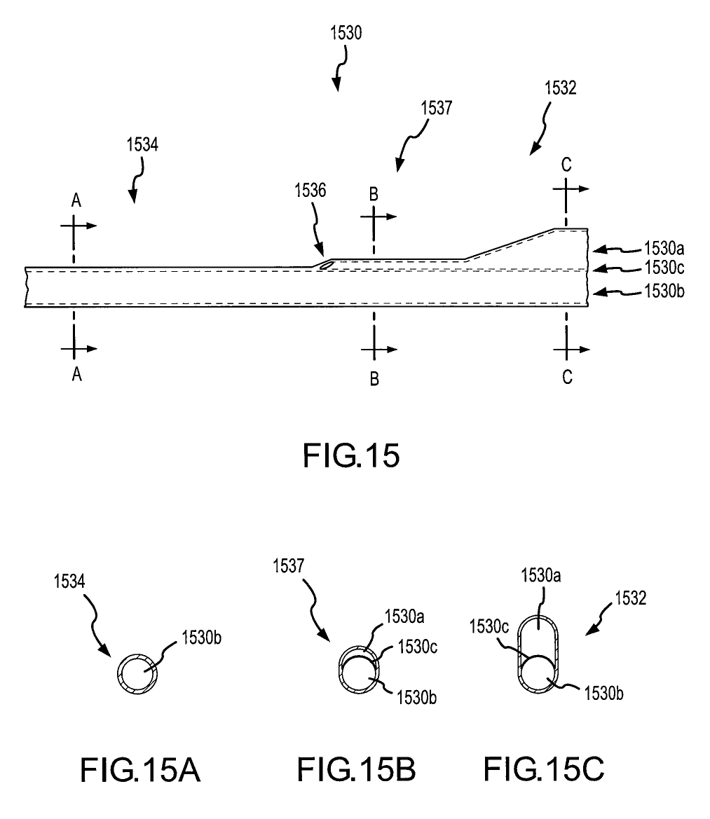

In some embodiments, the present sheath can be constructed using a flexible and lubricious separator between the two luminal spaces, which can be easily displaced as needed when devices are passed. Specifically, this device can have application in the same setting as devices as described in previously incorporated U.S. Provisional Patent Application Nos. 60/725,756 filed Oct. 11, 2005 and 60/742,579 filed Dec. 5, 2005. In an exemplary case, the sheath can be constructed with a dedicated 2-lumen hub, each lumen feeding into a separate lumen of the sheath. The sheath can taper to the desired diameter (10 F in this example) with the separator flap creating the two lumens. In this example, one of the lumens ends more proximally than the other, as is desired by the intended use of the product, as illustrated in FIGS. 15-15C. Introducer sheath 1530 includes a proximal section 1532, a distal section 1534, and a tapered transition section 1537 disposed therebetween, as depicted in FIG. 15. Transition section 1537 includes an exit port 1536. Introducer sheath 1530 also includes a first lumen 1530a configured to receive renal treatment system and a second lumen 1530b configured to receive a peripheral treatment system. Proximal section 1532 and transition section 1537 include a separator 1530c that divides first lumen 1530a and second lumen 1530b. Distal section 1534 includes a single lumen construction, as shown in FIG. 15A. Transition section 1537 includes a dual lumen construction with variable luminal cross-section, as shown in FIG. 15B. The cross sections of the first and second lumens may vary by movement of the wall separating the two lumens. Proximal section 1532 includes a dual lumen construction, where both lumens are “full size” to facilitate device entry, as shown in FIG. 15C. In some embodiments, separator 1530c includes a flexible separator flap.

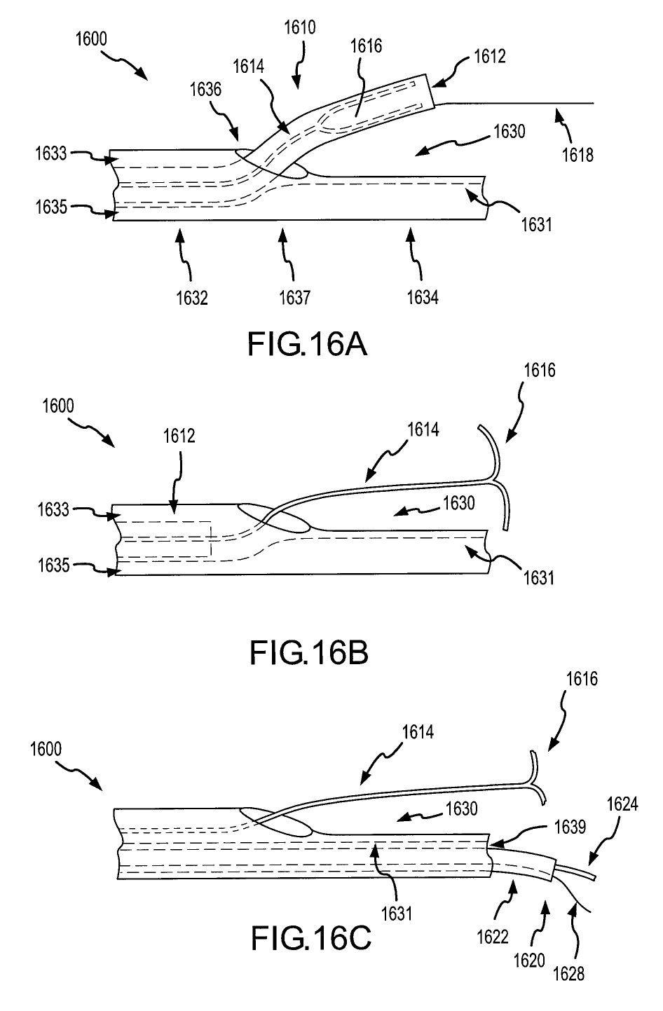

In some embodiments, a treatment sheath can be delivered over a guidewire through an introducer or main outer sheath to a site of renal artery cannulation. Once the guidewire is removed, the treatment catheter can be delivered through the treatment sheath. Upon delivery and deployment of the catheter, the treatment sheath can be retracted from the body to allow space for passage of a sheath used in a peripheral intervention. For example, as shown in FIGS. 16A-C, a treatment system 1600 can include an introducer sheath 1630, a renal treatment system 1610, and a peripheral treatment system 1620. Introducer sheath 1630 includes a proximal section 1632, a distal section 1634, and a transition section 1637 disposed therebetween. Transition section 1637 includes an exit port 1636. Introducer sheath 1630 also includes a flexible or stretchable separator 1631 that divides a first lumen 1633 from a second lumen 1635. Because separator 1631 is flexible, stretchable, or otherwise deformable, separator 1631 can move so as to allow variability in the interior dimensions of first lumen 1633 and second lumen 1635. Accordingly, first lumen 1633 and second lumen 1635 can be referred to as variable lumens, or as lumens having variable cross-sections or profiles. Renal treatment system 1610 can include a renal treatment sheath 1612, a renal catheter 1614 having a bifurcated distal end 1616, and a renal sheath guidewire 1618. Peripheral treatment system 1620 can include a peripheral treatment sheath 1622, a peripheral catheter 1624, and a peripheral sheath guidewire 1628.

FIG. 16A illustrates a method step that involves advancing renal treatment sheath 1612 along a renal sheath guidewire 1618 toward a renal treatment location. Renal treatment sheath 1612 passes through first lumen 1633 of the proximal section 1632 of introducer sheath 1630, and out of exit port 1636. Renal catheter 1614 is advanced through or housed within the interior of renal treatment sheath 1612 in a constrained or undeployed configuration. In some cases, due to the cross-sectional diameter of renal treatment sheath 1612, the section of separator 1631 that is within proximal section 1632 is flexed, stretched, or otherwise displaced, so as to accommodate renal treatment sheath 1612. Often, this involves increasing the cross-section of first lumen 1633 while decreasing the cross-section of second lumen 1635. When renal treatment sheath 1612 is placed in the desired position, renal sheath guidewire 1618 can be retracted. FIG. 16B illustrates a method step that encompasses retracting renal treatment sheath 1612 and deploying bifurcated distal end 116 of renal catheter 1614. Thereafter, renal treatment sheath 1612 can be removed via any of a variety of techniques as discussed elsewhere herein. FIG. 16C depicts a method step that involves advancing peripheral treatment sheath 1622 along a peripheral sheath guidewire 1628 toward a peripheral treatment location. Peripheral treatment sheath 1622 passes through second lumen 1635 of the proximal section 1632 and distal section 1634 of introducer sheath 1630, and out of distal port 1639. Peripheral catheter 1624 is advanced through or housed within the interior of peripheral treatment sheath 1622. In some cases, due to the cross-sectional diameter of peripheral treatment sheath 1622, the section of separator 1631 that is within proximal section 1632 is flexed, stretched, or otherwise displaced, so as to accommodate peripheral treatment sheath 1622, peripheral catheter 1624, or both. Often, this involves increasing the cross-section of second lumen 1635 while decreasing the cross-section of first lumen 1633. When peripheral treatment sheath 1622 is placed in the desired position, peripheral sheath guidewire 1628 can be retracted.

What is claimed is:

1. A system for delivering treatment to a renal artery and a peripheral artery, the system comprising:
a renal catheter;
a peripheral catheter; and
an introducer sheath having a first lumen and a second lumen;
wherein the first lumen is configured to receive the renal catheter and is sized to extend from a patient insertion site to a femoral or iliac artery location near or distal to a patient aortic branch, and the second lumen is configured to receive the peripheral catheter and is sized to extend from the patient insertion site to an opposite femoral or iliac artery location near or distal to the patient aortic branch.

2. The system according to claim 1, wherein at least one of the first lumen and the second lumen comprises a variable cross-section.

3. The system according to claim 1, wherein the introducer sheath comprises an exit port in communication with the first lumen.

4. The system according to claim 1, wherein the introducer sheath comprises a proximal section, a distal section, and a tapered section therebetween having an exit port.

5. The system according to claim 4, wherein the introducer sheath comprises a coil disposed at the proximal section, the distal section, and the tapered section, and the coil has a first pitch at the proximal and distal sections and a second pitch at the tapered section.

6. The system according to claim 4, wherein the exit port comprises a plug having a slit.

7. The system according to claim 1, further comprising a drug infusion source in operative association with the renal catheter.

8. A system for delivering treatment to a renal artery and a peripheral artery, the system comprising:
a renal catheter having a distal bifurcation;
a peripheral catheter; and
an introducer sheath having a first lumen and a second lumen;
wherein the first lumen is configured to receive the renal catheter and is separated from the second lumen by a flexible separator flap, and the second lumen is configured to receive the peripheral catheter.

9. The system according to claim 8, wherein at least one of the first lumen and the second lumen comprises a variable cross-section.

10. The system according to claim 8, wherein the introducer sheath comprises an exit port in communication with the first lumen.

11. The system according to claim 8, wherein the introducer sheath comprises a proximal section, a distal section, and a tapered section therebetween having an exit port.

12. The system according to claim 11, wherein the introducer sheath comprises a coil disposed at the proximal section, the distal section, and the tapered section, and the coil has a first pitch at the proximal and distal sections and a second pitch at the tapered section.

13. The system according to claim 8, further comprising a drug source in operative association with the renal catheter.

14. A method of delivering treatment to a renal artery and a peripheral artery, the method comprising:
positioning an introducer sheath in an iliac artery;
advancing a renal catheter through a first lumen of the introducer sheath to a location at or near the renal artery; and
advancing a peripheral catheter through a second lumen of the introducer sheath to a location at or near the peripheral artery;
wherein the first lumen and the second lumen of the introducer sheath are separated by a flexible separator flap.

15. The method according to claim 14, wherein at least one of the first lumen and the second lumen comprises a variable cross-section.

16. The method according to claim 14, further comprising advancing the renal catheter through an exit port of the introducer sheath, where the exit port is in communication with the first lumen.

17. The method according to claim 14, further comprising providing a renal treatment via the renal catheter, and providing a peripheral treatment via the peripheral catheter.

18. The method according to claim 14, further comprising delivering a renal therapeutic agent via the renal catheter, wherein the renal therapeutic agent improves kidney function, prevents kidney damage, or both.

19. A system for delivering treatment to a renal artery and a peripheral artery, the system comprising:
a renal catheter;
a peripheral catheter; and
an introducer sheath having an exit port;
wherein the introducer sheath is configured to receive the renal catheter and the peripheral catheter and is sized to extend from a patient insertion site past a femoral or iliac artery location near or distal to a patient aortic branch to an opposite femoral or iliac artery location near or distal to the patient aortic branch, such that when the introducer sheath is placed in the patient, the exit port is situated at or near the a femoral or iliac artery location near or distal to the patient aortic branch.

20. The system according to claim 18, wherein the introducer sheath comprises a proximal section, a distal section, a tapered section therebetween, and the exit port is disposed at the tapered section.

21. The system according to claim 19, wherein the introducer sheath comprises a coil disposed at the proximal section, the distal section, and the tapered section, and the coil has a first pitch at the proximal and distal sections and a second pitch at the tapered section.

22. The system according to claim 18, wherein the renal catheter comprises a bifurcated distal end.

23. A method of positioning a renal treatment system and a peripheral treatment system, the method comprising:
positioning an introducer sheath in an iliac artery;
advancing a renal catheter of the renal treatment system through a lumen of the introducer sheath to a location at or near a renal artery; and
advancing a peripheral catheter of the peripheral treatment system through the lumen of the introducer sheath to a location at or near a peripheral artery.

24. The method according to claim 23, further comprising advancing the renal catheter through an exit port of the introducer sheath, wherein the exit port is located proximal to a distal end of the introducer sheath.

25. The method according to claim 23, further comprising providing a renal treatment via the renal catheter, and providing a peripheral treatment via the peripheral catheter.

26. The method according to claim 23, further comprising delivering a renal therapeutic agent via the renal catheter, wherein the renal therapeutic agent improves kidney function, prevents kidney damage, or both.

27. A system for delivering treatment to a renal artery and a peripheral artery, the system comprising:
a plurality of renal catheters;
a plurality of peripheral catheters; and
an introducer sheath having a plurality of renal catheter lumens and a plurality of peripheral catheter lumens;
wherein each of the plurality of renal catheter lumens is configured to receive at least one of the plurality of renal catheters and is sized to extend from a patient insertion site to a femoral or iliac artery location near or distal to a patient aortic branch, and each of the plurality of peripheral catheter lumens is sized to extend from the patient insertion site to an opposite femoral or iliac artery location near or distal to the patient aortic branch.

28. The system according to claim 27, wherein each of the plurality of renal catheter lumens and peripheral catheter lumens exit the introducer sheath at distinct locations along a length of the sheath.

29. The system according to claim 27, wherein at least one lumen of the plurality of renal catheter lumens and the plurality of peripheral catheter lumens comprises a variable cross-section.

30. A system for delivering treatment to a renal artery and a peripheral artery, the system comprising:
a plurality of renal catheters, wherein at least one of the renal catheters comprises a distal bifurcation;
a plurality of peripheral catheters; and
an introducer sheath having a plurality of renal catheter lumens and a plurality of peripheral catheter lumens;
wherein each of the plurality of renal catheter lumens is configured to receive at least one of the plurality of renal catheters, each of the plurality of peripheral catheter lumens is configured to receive at least one of the plurality of peripheral catheters, and at least one of the renal catheter lumens is separated from at least one of the peripheral catheter lumens by a flexible separator flap.

31. The system according to claim 30, wherein each of the plurality of renal catheter lumens and peripheral catheter lumens exit the introducer sheath at distinct locations along a length of the sheath.

32. The system according to claim 30, wherein at least one lumen of the plurality of renal catheter lumens and the plurality of peripheral catheter lumens comprises a variable cross-section.

33. A method of delivering treatment to a renal artery and a peripheral artery, the method comprising:
positioning an introducer sheath in an iliac artery, the introducer sheath having a plurality of renal catheter lumens and a plurality of peripheral catheter lumens;
advancing each of a plurality of renal catheters through a respective renal catheter lumen of the plurality of renal catheter lumens to a location at or near the renal artery; and
advancing each of a plurality of peripheral catheters through a respective peripheral catheter lumen of the plurality of peripheral catheter lumens to a location at or near the peripheral artery;
wherein at least one of the renal catheter lumens is separated from at least one of the peripheral catheter lumens by a flexible separator flap.

34. The method according to claim 33, wherein each of the plurality of renal catheter lumens and peripheral catheter lumens exit the introducer sheath at distinct locations along a length of the sheath.

35. The method according to claim 33, wherein at least one lumen of the plurality of renal catheter lumens and the plurality of peripheral catheter lumens comprises a variable cross-section.

36. The method according to claim 33, further comprising providing a renal treatment via at least one of the plurality of renal catheters, and providing a peripheral treatment via at least one of the plurality of peripheral catheters.

37. The method according to claim 33, further comprising delivering a renal therapeutic agent via at least one of the plurality of renal catheters, wherein the renal therapeutic agent improves kidney function, prevents kidney damage, or both.
